(19) United States
(12) Patent Application Publication
(10) Pub. No.: US 2025/0143573 A1
(43) Pub. Date: May 8, 2025

(54) SPAWN A MESH NETWORK IN RESPONSE TO A MEDICAL EVENT

(71) Applicant: Medtronic, Inc., Minneapolis, MN (US)

(72) Inventors: Christopher D. Koch, Minneapolis, MN (US); Christopher T. House, Pine Island, MN (US); Laura J. Philippsen, St. Paul, MN (US); Grant A. Neitzell, Loretto, MN (US); Abhijit P. Jejurkar, Plymouth, MN (US); Paul G. Krause, Mahtomedi, MN (US)

(21) Appl. No.: 18/837,326

(22) PCT Filed: Jan. 30, 2023
(86) PCT No.: PCT/IB2023/050804

§ 371 Date: Aug. 9, 2024

Related U.S. Application Data
(60) Provisional application No. 63/267,826, filed on Feb. 10, 2022.

(51) Int. Cl. A61B 5/00; G16H 10/60; G16H 40/67

(52) U.S. Cl. CPC A61B 5/002; A61B 5/7282; G16H 10/60; G16H 40/67

(57) ABSTRACT

A medical includes a first device configured to receive data from a medical device, determine based on the data that the patient is experiencing an acute health event, and in response to determining that the patient is experiencing the acute heath event, broadcast a message to a plurality of computing devices. The plurality of devices includes a second device configured to receive the message from the first device and establish a communication session with the first device in response to receiving the message.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect acute health events based on the physiological signals, such as episodes of cardiac arrhythmia, myocardial infarction, stroke, or seizure. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events are associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes techniques for establishing a mesh network, peer-to-peer network, or other such communication session between two or more proximate devices in response to a medical device detecting an acute health event.

According to one example, a device includes communication circuitry configured to communicate between a medical device of a patient and a plurality of computing devices and processing circuitry communicatively coupled to the communication circuitry and configured to: receive, via the communication circuitry, data from the medical device; determine based on the data that the patient is experiencing an acute health event; and in response to determining that the patient is experiencing the acute heath event, broadcast a message to the plurality of computing devices to establish a wireless network with one or more of the plurality of computing devices.

According to another example, a method includes receiving data from a medical device; determining based on the data that a patient is experiencing an acute health event; and in response to determining that the patient is experiencing the acute heath event, broadcast a message to the plurality of computing devices to establish a wireless network with one or more of the plurality of computing devices.

According to another example, a computer readable medium stores instructions that when executed by one or more processors causes the one or more process to perform any of the various techniques described herein.

According to another example, a system includes a first device configured to receive data from a medical device; determine based on the data that the patient is experiencing an acute health event; and in response to determining that the patient is experiencing the acute heath event, broadcast a message to a plurality of computing devices. The system also includes a second device, wherein the second device belongs to the plurality of devices and the second device is configured to receive the message from the first device; and establish a communication session with the first device in response to receiving the message.

According to another example, a first is device configured to communicate with a second device, and the first device includes communication circuitry configured to receive a message from the second device and processing circuitry communicatively coupled to the communication circuitry and configured to receive, via the communication circuitry, data from the medical device; determine based on the data that the patient is experiencing an acute health event; and in response to receiving the message, establish a wireless network for transmission of medical information with the second device.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured detect acute health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flowchart illustrating examples of the techniques of the present disclosure.

Like reference characters refer to like elements throughout the figures and description.

### DETAILED DESCRIPTION

A patient with a medical device may have an acute health event, such as sudden cardiac arrest, stroke, acute myocardial infarction, or anaphylactic shock, and become incapacitated. In some systems, a medical device may detect the acute health event in the patient and send a notification of the acute health event to a computing device of the patient, such as a phone or smart watch. The computing device may then contact a first responder via a network connection through WiFi or a cellular network.

An acute health event could, however, occur while a patient is in flight, at sea, or otherwise in a structure or location where network access is unavailable. Without network access, the computing device of the patient may not be able to contact an emergency service, such as 911, to obtain medical help. This disclosure describes techniques for configuring a computing device of the patient to spawn a mesh network, peer-to-peer network, or otherwise communicate with nearby computing devices to alert users of the nearby computing devices that the patient is experiencing the acute health event. The computing device of the patient may additionally or alternatively use one or more of the nearby computing devices as a gateway device for obtaining network access to public or private networks.

In one example use case of the techniques of this disclosure, a medical device of a patient may detect an acute health event while the patient is in flight. The medical device may send, via a Bluetooth protocol or WiFi protocol for example, a notification to a computing device of the patient that the patient is experiencing the acute health event, but due to being in flight, the computing device may not have a cellular or Internet connection available to alert an emergency service. In such a case, the computing device of the patient may broadcast, via Bluetooth or WiFi for example, a message indicating that the patient has experienced or is experiencing the acute health event.

Another computing device may receive the message. The other computing device may, for example, include an application or software given to first responders, or other users that may be capable of assisting the patient, and that software or application may configure the other computing device to receive and process the message. In the particular case of a patient being in flight, the other computing device may, for instance, belong to a flight attendant who is trained to perform CPR and use a defibrillator, and in response to receiving the message, the other computing device may generate a notification to alert the user of the other computing device, e.g., the flight attendant, to the acute health event. In other examples, the other computing device may belong to an off duty paramedic, doctor, or anyone else trained and willing to provide emergency first aid or otherwise willing to allow for their personal computing device to be used as a gateway device for network access. The other device may additionally or alternatively relay information included in the message to an emergency service using a network connection of the other computing device. In some examples, the message may include information, such as an SSID of the patient computing device, that can be used by the other computing device to establish a communication session between the patient computing device and the other computing device, and thus give the patient computing device network access using the other computing device as a gateway.

Although the above example describes a scenario where a patient computing device lacks network access, various techniques of this disclosure may also be used in scenarios where the patient computing device has network access. For example, while at a shopping mall or an arena, a medical device of a patient may detect an acute health event. The medical device may send, e.g., via Bluetooth or WiFi, a notification to a computing device of the patient that the patient is experiencing the acute health event, and the computing device of the patient may contact an emergency service, such as 911, to obtain medical help. The computing device of the patient may also broadcast, via Bluetooth or WiFi for example, a message indicating that the patient has experienced or is experiencing the acute health event. Another computing device nearby the patient computing device may receive the message and generate a notification that the patient is experiencing the acute health event. The other computing device may additionally or alternatively send a notification, over a local area network or peer-to-peer network for example, to an emergency responder at the mall or arena to alert the emergency responder to the acute health event of the patient. The other computing device may act as a gateway device for the patient computing device to access the local area network. In such a case, the nearby emergency responder may be able to reach the patient more quickly than the emergency service contact by the patient computing device.

A variety of types of implantable and computing devices are configured detect arrhythmia episodes and other acute health events based on sensed ECGs and, in some cases, other physiological signals. Computing devices that may be used to non-invasively sense and monitor ECGs and other physiological signals include wearable devices with electrodes configured to contact the skin of the patient, such as patches, watches, or necklaces, and other non-contact monitoring devices, such as devices configured to monitor sound, radar, light, images, etc. Such computing devices may facilitate relatively longer-term monitoring of patient health during normal daily activities.

Implantable medical devices (IMDs) also sense and monitor ECGs and other physiological signals, and detect acute health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ Insertable Cardiac Monitor (ICM) or the LINQ II™ ICM, available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

Such an IMD may interact with other devices. The IMD may, for example, sense an indication of an acute health event of the patient and communicate the detection of the acute health event to another device so that the other device can send an alert or notification regarding the acute health event, such as call an emergency service. These other devices may in some instances also be verification devices or part of a verification system and may be configured to verify whether the patient actually experienced the sensed acute health event. For example, the verification device or system may verify the acute health event, and based on the verification of the acute health event, send the alert regarding the acute health event. The verification device or system may include at least one of a smartphone, wearable device, tablet device, or other such computing device.

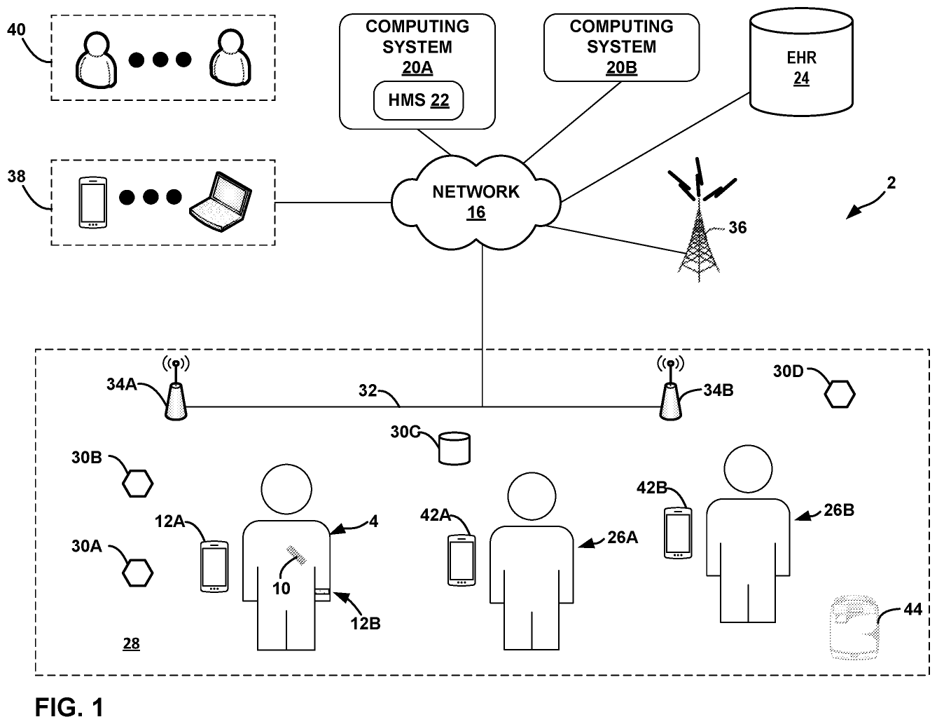

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. The example techniques may be used with one or more patient sensing devices, e.g., IMD 10, which may be in wireless communication with one or more patient computing devices, e.g., patient computing devices 12A and 12B (collectively, “computing devices 12”). Computing devices 12 may be verification devices and may attempt to verify the occurrence of an acute health event. Although not illustrated in FIG. 1, IMD 10 include electrodes and other sensors to sense physiological signals of patient 4, and may collect and store sensed physiological data based on the signals and detect episodes based on the data.

In some examples, although not depicted in FIG. 1, patient 4 may have a plurality of patient sensing devices, such as IMD 10. In some examples, the plurality of patient sensing devices may communicate with each other and/or computing device(s) 12. In some examples, the plurality of patient sensing devices may use time matching techniques, such determining a difference in a clock of each patient sensing device and applying the difference when saving a time of a sensed indication of an acute health event, or use a common clock. In this manner, sensed indications of acute health events of different patient sensing devices may be synchronized. In another example, one patient sensing device may be configured as a master and other patient sensing devices may be configured as slaves.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, drug pumps, wearable devices, or other such devices. Furthermore, although described primarily in the context of examples including a single implanted patient sensing device, in some examples a system includes one or more patient sensing devices, which may be implanted within patient 4 or external to (e.g., worn by) patient 4.

The techniques and systems of this disclosure may be implemented in an implantable medical device that can continuously and/or periodically sense parameters of a patient without human intervention while subcutaneously implanted in a patient over months or years and perform millions of operations per second on patient sensor data to identify an acute health event. Using techniques of this disclosure in an implantable medical device may be advantageous when a physician cannot be continuously present with the patient over weeks or months to evaluate sensor data and/or where performing millions of operations on weeks or months of sensor data could not practically be performed in the mind of a physician with techniques of this disclosure.

IMD 10 may take the form of an elongated rectangular prism (e.g., LINQ II™ ICM, available from Medtronic plc) having rounded corners and a rounded distal end portion as the rounded distal end of the device assists in allowing it to advance into body tissue, providing blunt dissection of the tissue as it advances. IMD 10 may have length (L), e.g., from a proximal end to distal end, width (W) and depth (D) as illustrated. In this particular embodiment, the width is greater than the depth, providing radial asymmetry along the longitudinal axis of the device and assisting in maintaining the IMD 10 in its proper orientation with an upper surface facing outward after being injected. In some examples, IMD 10 may be injected with other orientations as well. In some examples, IMD may include projections to prevent longitudinal and/or rotational movement of the device after being injected. In some examples, IMD 10 may have a length less than 5 centimeters (cm), a width less than 1 cm, a depth less than 0.5 cm, and a volume of less than 1.5 cubic centimeters (cm³). For example, IMD 10 may have a length of 45.1 millimeters (mm), a width of 8 mm, a depth of 4.2 mm, and a volume of 1.4 cm³. In other examples, IMD 10 may have a length less than 46 mm, a width less than 4 mm, a depth less than 2 mm, and a volume of less than or equal to 0.25 cm³. For example, IMD 10 may have a length from 30 mm to 45 mm, a width less than 4 mm, a depth less than 2 mm, and a volume of less than or equal to 0.25 cm³.

The example of FIG. 1 includes environment 28. Environment 28 may be a home, office, place of business, or public venue, where access to communications networks such as WiFi or a cellular network are readily available. Environment 28 may also be a setting where access to a WiFi or a cellular network is limited, such as an airplane in flight, a ship out to sea, or in a basement of a building.

Computing devices 12 are configured for wireless communication with IMD 10. Computing devices 12 retrieve or receive event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing devices 12 may communicate with IMD 10 and each other according to the Bluetooth®, Bluetooth® Low Energy (BLE), or other wireless communication protocols, as examples. In some examples, only one of computing devices 12, e.g., computing device 12A, is configured for communication with IMD 10, e.g., due to execution of software (e.g., part of a health monitoring application as described herein) enabling communication and interaction with an IMD. In some examples, computing device(s) 12 may be configured to receive an indication of an acute health event of patient 4 from IMD 10.

In some examples, computing device(s) 12, e.g., wearable computing device 12B in the example illustrated by FIG. 1A, may include electrodes and other sensors to sense physiological signals of patient 4, and may collect and store physiological data and detect health events based on such signals. Computing device 12B may be incorporated into the apparel of patient 14, such as within clothing, shoes, eyeglasses, a watch, ring, necklace, wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilities detection of acute health events of patient 4 by system 2, and the responses of system 2 to such acute health events.

Computing device(s) 12 may transmit data, including data retrieved or received from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to detect acute health events of patient 4 based on data sensed by IMD 10 and, in some cases, other data, such as data sensed by computing devices 12A and/or 12B, and data from EHR 24. In response to detection of an acute health event, IMD 10 may wirelessly transmit an indication of the acute health event, such as a message, to one or both of computing devices 12A and 12B. The message may indicate that IMD 10 detected an acute health event of patient 4. The message may indicate a time that IMD 10 detected the acute health event. The message may include physiological data collected by IMD 10, e.g., data which lead to detection of the acute health event, data prior to detection of the acute health event, and/or real-time or more recent data collected after detection of the acute health event. The physiological data may include values of one or more physiological parameters and/or digitized physiological signals. Some examples of acute health events are cardiac arrest, ventricular fibrillation, ventricular tachycardia, myocardial infarction, pause in heat rhythm (asystole), pulseless electrical activity (PEA), acute respiratory distress syndrome (ARDS), stroke, seizure, fall, anaphylactic shock, or respiratory failure.

In some examples, in response to the message indicative of the sensing of the acute health event from IMD 10, computing device(s) 12 may prompt patient 4 to provide a response. For example, computing device(s) 12 may audibly ask the patient if they are okay or may ask the patient to provide tactile input indicating that they are okay in an attempt to elicit a response. Computing device(s) 12 may wait for a response from patient 4. After a predetermined period of time, which may be on the order of up to 60 seconds, if the patient had not responded, computing device(s) 12 may attempt to verify the acute health event. For example, computing device 12B may attempt to take a pulse of patient 4.

In some examples, in response to the message from IMD 10, computing device(s) 12 may also output an alarm that may be visual and/or audible, and configured to immediately attract the attention of patient 4 or any person in environment 28 with patient 4, e.g., bystanders 26A and 26B (collectively bystanders 26). Computing device(s) 12 may also transmit a message to HMS 22 via network 16. The message may include the data received from IMD 10 and, in some cases, additional data collected by computing device(s) 12 or other devices in response to the detection of the acute health event by IMD 10. For example, the message may include a location of patient 4 determined by computing device(s) 12.

Environment 28 includes computing facilities, e.g., a local network 32, by which IMD 10, computing devices 12, devices 30, and other devices within environment 28 may communicate via network 16, e.g., with HMS 22 or with other devices on local network 32. For example, environment 28 may be configured with wireless technology, such as 802.11 wireless networks, 802.15 ZigBee networks, an ultrawideband protocol, near-filed communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, IMD 10, computing devices 12, devices 30, and other devices within environment 28 may be configured to communicate with network 16, e.g., with HMS 22, via a cellular base station 36 and a cellular network.

Computing device(s) 12 may include input devices and interfaces to allow a user to override the alarm in the event the detection of the acute health event by IMD 10 was false. In some examples, one or more of computing device(s) 12 may implement an event assistant. The event assistant may provide a conversational interface or a tactile interface for patient 4 and/or bystanders 26 to exchange information with the computing device or another device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user.

In some examples, computing device(s) 12 and/or HMS 22 may implement one or more algorithms to evaluate the sensed physiological data received from IMD 10, and in some cases additional physiological or other data sensed or otherwise collected by the computing device(s), to confirm or override the detection of the acute health event by IMD 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event.

In examples in which computing device(s) 12 are configured to perform an acute health event confirmation analysis or verification, computing device(s) 12 may transmit alert messages to HMS 22 in response to confirming or verifying the acute health event. In some examples, computing device(s) 12 may be configured to transmit the alert messages prior to completing the confirmation or verification analysis, and transmit cancellation messages in response to the analysis overriding the detection of the acute health event by IMD 10. HMS 22 may be configured to perform a number of operations in response to receiving an alert message from computing device(s) 12 and/or device(s) 30. HMS 22 may be configured to cancel such operations in response to receiving a cancellation message from computing device(s).

In some examples, computing device(s) 12 may transmit the alert to a care provider, an emergency medical technician, or other designated persons in environment 28 or near environment 28. For example, the alert may be a communication to the emergency medical technician, or local neighborhood alert system with an automated emergency defibrillator service, to a care provider, etc. In some examples, the alert includes collected data from IMD 10 and the verification device or system, such that medical personnel may be prepared to take quick action on arrival in environment 28. In some examples, the alert includes at least one of a telephone call, a short message service message, an email, a web alert, a security system alert, a social media alert, an audible alert, or a visual alert.

In some examples, the verification device or system may send an alert through a security system in environment 28 to flash a “save our souls” (SOS) message, sound an audible alarm, or the like. In some examples, the verification device or system may notify neighbors of patient 4 of a medical emergency. In some examples, the verification system may send an alarm or warning to everyone and every device around the environment 28.

For example, HMS 22 may be configured to transmit alert messages to one or more computing devices 38 associated with one or more care providers 40 via network 16. Care providers may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, including sensed physiological parameters, time of the acute health event, location of patient 4, and results of the analysis by IMD 10, computing device(s) 12, and/or HMS 22. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or devices 30 may be configured to automatically contact EMS, e.g., autodial 911 (e.g., in the United States or North America to use the telephone system to contact a 911 call center), in response to receiving an alert message from IMD 10. Again, such operations may be cancelled by patient 4, bystanders 26, or another user via a user interface of computing device(s) 12 or device(s) 30, or automatically cancelled by computing device(s) 12 based on a confirmatory analysis or verification performed by the computing device(s) overriding the detection of the acute health event by IMD 10.

Similarly, HMS 22 may be configured to transmit an alert message to computing devices 42A and 42B (collectively computing devices 42) of bystanders 26, which may improve the timeliness and effectiveness of treatment of the acute health event of patient 4 by bystanders 26. Computing devices 42 may be similar to computing devices 12 and computing devices 38, e.g., a smartphone. In some examples, HMS 22 may determine that bystanders 26 are proximate to patient 4 based on a location of patient 4, e.g., received from computing device(s) 12, and locations of computing devices 42, e.g., reported to HMS 22 by an application implemented on computing devices 42. In some examples, HMS 22 may transmit the alert message to any of computing devices 42 in an alert area determined based on the location of patient 4, e.g., by transmitting the alert message to all computing devices in communication with base station 36.

In some examples, the alert message to bystanders 26 may be configured to assist a layperson in treating patient. For example, the alert message to bystanders 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an automated external defibrillator (AED) 44 or life vest, and instructions for use of the equipment. In some examples, computing device(s) 12, device(s) 30, and/or computing devices 42 may implement an event assistant configured to use natural language processing and context data to provide a conversational interface for bystanders 26. The assistant may provide bystanders 26 with directions for providing care to patient 4, and respond to queries from bystanders 26 about how to provide care to patient 4.

In some examples, HMS 22 may mediate bi-directional audio (and in some cases video) communication between care providers 40 and patient 4 or bystanders 26. Such communication may allow care providers 40 to evaluate the condition of patient 4, e.g., through communication with patient 4 or bystanders 26, or through use of a camera or other sensors of the computing device or device, in advance of the time they will begin caring for the patient, which may improve the efficacy of care delivered to the patient. Such communication may also allow the care providers to instruct bystanders 26 regarding first responder treatment of patient 4.

Thus far, the functionality of computing devices 12 has largely been described in the context of computing devices 12 having access to network 16 and local network 32. In some use cases, however, computing devices 12 may not have access to network 16 and local network 32. In such situations, if computing devices 12 receive data from IMD 10 indicating that patient 4 is experiencing an acute health event, then computing devices 12 may be configured to broadcast a message to a plurality of computing devices, including computing devices 42, indicating that patient 4 is experiencing the acute health event. Computing devices 12 may, for example, broadcast this message using a Bluetooth protocol or WiFi protocol. Computing devices 42 may be configured to listen for this message, and in response to receiving the message, establish a communication session with computing devices 12. Computing devices 12 may then use one of computing devices 42 as a gateway to access network 16 or local network 32. That is, the functionality described above for computing devices 12 may be performed using one of computing devices 42 as a gateway to local network 32 or network 16 rather than with direct access to local network 32 or network 16.

In various implementations of the techniques described herein, computing device 12 may be configured to broadcast the message to a variety of different ranges, such as within a range of less than 150 meters of computing device 12, within a range of less than 40 meters of computing device 12, within a range of less than 275 meters of computing device 12, within a range of less than 125 meters of computing device 12, within a range of at least 15 meters of computing device 12, or within a range of 1-100 meters of computing device 12.

In some instances, computing devices 12 may be configured to transmit encrypted medical information to one of computing devices 42, and then computing devices 42 may relay the encrypted medical information to a third party using one or both of network 16 or local network 32. Computing devices 42 may be configured to relay the encrypted medical information without decrypting the medical information, and in fact, computing devices 42 may not even be capable of decrypting the medical information, thus protecting the privacy of patient 4. Computing devices 42 may additionally alert bystanders 26 that patient 4 is experiencing the acute health event.

In one example, to facilitate establishment of the communication session, computing device 12 may broadcast a Bluetooth pairing request with a specific universally unique identifier (UUID). Computing devices 42 may be configured to listen for Bluetooth pairing requests and associate that specific UUID with the presence of an acute health event occurring nearby. Thus, in response to detecting the specific UUID, computing devices 42 may launch certain applications or software modules that are configured to facilitate the transmission of data from computing device 12 over local network 32 and network 16 using one of computing devices 42 as a gateway device.

Similarly, to facilitate establishment of the communication session, computing device 12 may broadcast a WiFi pairing request with a specific service set identifier (SSIDs) or other unique identifier. Computing devices 42 may be configured to detect SSIDs and associate that specific SSID with the presence of an acute health event occurring nearby. Thus, in response to detecting the specific SSID, computing devices 42 may launch certain applications or software modules that are configured to facilitate the transmission of data from computing device 12 over local network 32 and network 16 using one of computing devices 42 as a gateway device.

Whether using Bluetooth or WiFI, different types of identifiers may be used to broadcast the need for an emergency network. Moreover, before establishing a communication session with computing devices 12, computing devices 42 may be configured to perform additional verifications to confirm that the computing device is not spoofing the UUID, SSID, or other type of identifier.

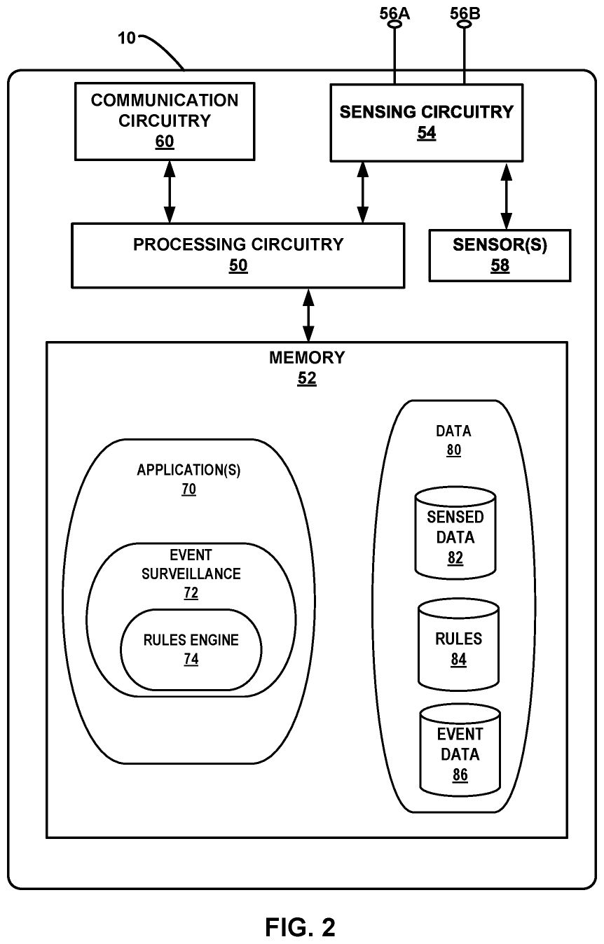

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes sensing circuitry 58, such as one or more accelerometers, microphones, optical sensors, temperature sensors, and/or pressure sensors. In some examples, sensing circuitry 54 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include an acute health event surveillance application 72. Processing circuitry 50 may execute event surveillance application 72 to detect an acute health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning.

As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart 6 of patient 4 (FIG. 1). In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects an acute health event, event surveillance application 72 may store the sensed data 82 that lead to the detection (and in some cases a window of data preceding and/or following the detection) as event data 86.

In some examples, in response to detection of an acute health event, processing circuitry 50 transmits, via communication circuitry 60, event data 86 for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or devices 30. In response to receiving the message, computing device(s) 12 may attempt to elicit a response from patient 4 as discussed above with respect to FIG. 1.

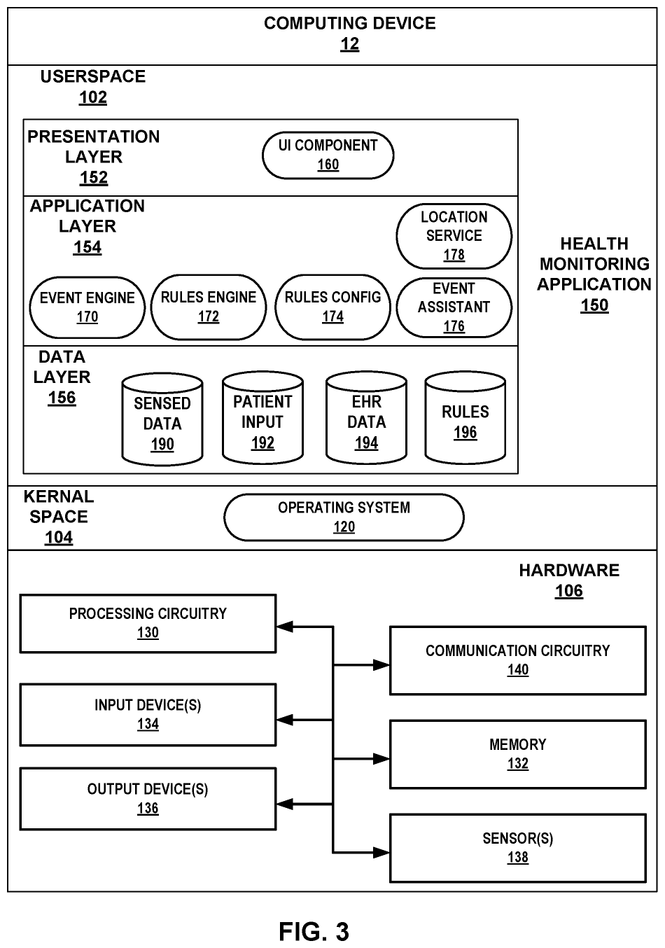

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a smart speaker, a laptop, a tablet computer, a personal digital assistant (PDA), a smartwatch or other wearable computing device. In some examples, devices 42 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, sensing circuitry 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 136 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

Sensing circuitry 138 of computing device 12 may sense physiological parameters or signals of patient 4. Sensor(s) 138 may include electrodes, 3-axis accelerometers, an optical sensor, impedance sensors, temperature sensors, pressure sensors, heart sounds sensors, and other sensors, and sensing circuitry (e.g., including an ADC), similar to those described above with respect to IMD 10 and FIG. 2.

Communication circuitry 140 of computing device 12 may communicate with other devices by transmitting and receiving data. For example, communication circuitry 140 may be configured to communicate with a medical device of a patient and a plurality of computing devices. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 170 may be responsive to receipt of an alert transmission from IMD 10 indicating that IMD 10 detected an acute health event. Event engine 170 may control performance of any of the operations in response to detection of an acute health event ascribed herein to computing device 12, such as activating an alarm, transmitting alert messages to HMS 22, controlling devices 30, and analyzing data to confirm or override the detection of the acute health event by IMD 10.

Rules engine 172 analyzes sensed data 190, and in some examples, patient input 192 and/or EHR data 194, to determine whether there is a sufficient likelihood that patient 4 is experiencing the acute health event detected by IMD 10 or to verify that patient 4 has experienced the acute health event detected by IMD 10. Sensed data 190 may include data received from IMD 10 as part of the alert transmission, additional data transmitted from IMD 10, e.g., in “real-time,” and physiological parameters and other data related to the condition of patient 4 collected by computing device(s) 12, devices 42, or some other such device. Rules engine 172 may determine whether the physiological parameters meet predefined criteria to verify the acute health event. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystanders 26. The queries and responses may occur responsive to the detection of the event by IMD 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Rules engine 172 may apply rules 196 to the data. Rules 196 may include one or more models, algorithms, decision trees, and/or thresholds. In some cases, rules 196 may be developed based on machine learning. In some examples, rules 196 and the operation of rules engine 172 may provide a more complex analysis of the sensed data received from IMD 10, than is provided by rules engine 74 and rules 84. In some examples, rules 196 include one or more models developed by machine learning, and rules engine 172 applies feature vectors derived from the data to the model(s). For example, rules engine 172 may apply rules 196 to determine whether the physiological parameters captured by IMD 10 and the verification device or system meet predefined criteria to verify the acute health event.

Rules configuration component 174 may be configured to modify rules 196 (and in some examples rules 84) based on feedback indicating whether the detections and confirmations of acute health events by IMD 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

As discussed above, event assistant 176 may provide a conversational interface or tactile interface for patient 4 and/or bystanders 26 to exchange information with computing device 12. Event assistant 176 may query the user regarding the condition of patient 4 in response to receiving the alert message from IMD 10. Responses from the user may be included as patient input 192. Event assistant 176 may use natural language processing and context data to interpret utterances by the user. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystanders 26.

Location service 178 may determine the location of computing device 12 and, thereby, the presumed location of patient 4. Location service 178 may use global position system (GPS) data, multilateration, and/or any other known techniques for locating computing devices. In some examples, location service 178 may utilize data from other devices to determine the location of patient 4, such as devices 42. For example, data from a camera, a radar system, a sonar system, or a lidar system may be used to determine where in environment 28 (FIG. 1) patient 4 is located. In some examples, location service 178 may track where patient 4 is during different times of the day and use the most frequent location at the time of the day that the indication of the acute medical event was sensed as a starting position to determine the location of patient 4. For example, location service 178 may employ one or more devices, such as devices 42, to check to see if patient 4 is located at the most frequent location for that time of day.

As disclosed herein, processing circuitry 130 may be configured to receive, via communication circuitry 140, data from IMD 10, and determine based on the data that patient 4 is experiencing an acute health event. Health monitoring application 150 may, for example, make or confirm the determination that patient 4 is experiencing the acuate health event. In response to determining that patient 4 is experiencing the acute heath event, processing circuitry 130 may cause communications circuitry 140 to broadcast a message to a plurality of computing devices, such as computing devices 42, to establish an ad hoc, wireless network with one or more of the plurality of computing devices. Processing circuitry 130 may, for example, determine that a network connection, such as an Internet connection or a cellular network connection, is unavailable and cause communications circuitry 140 to broadcast the message to the plurality of computing devices in response to determining that the network connection is unavailable. As one example, the message may be a Bluetooth pairing request that includes a known UUID or an identifier for a WiFi network, and the plurality of computing devices may have a priori knowledge that the identifier, e.g., the UUID or SSID, is intended for emergency communication sessions for the purpose of relaying medical information. That is the identifier may be known to one of the plurality of computing devices as an identifier that is specially designated for emergency communication sessions for the purpose of relaying medical information.

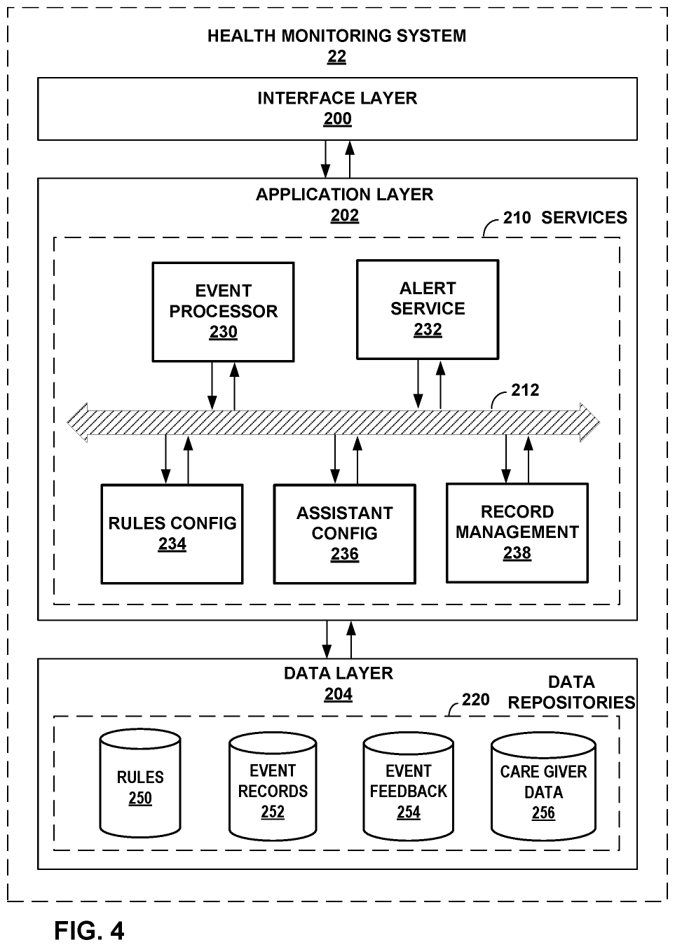

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12 and 48 may operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or device(s) 30 indicating that IMD 10 detected an acute health event of patient and, in some examples, that the transmitting device confirmed or verified the detection. Event processor service 230 may initiate performance of any of the operations in response to detection of an acute health event ascribed herein to HMS 22, such as communicating with patient 4, bystanders 26, and care providers 40, activating the verification device or system (e.g., computing device(s) 12 or 42) and, in some cases, analyzing data to confirm or override the detection of the acute health event by IMD 10. In some examples, rather than actually verifying the acute health event, the verification device or system may transmit the sensed physiological parameters to HMS 22 and HMS 22 may verify the acute health event.

Record management service 238 may store the patient data included in a received alert message within event records 252. Alert service 232 may package the some or all of the data from the event record, in some cases with additional information as described herein, into one more alert messages for transmission to bystanders 26 and/or care providers 40. Care provider data 256 may store data used by alert service 232 to identify to whom to send alerts based on locations of potential bystanders 26 and care providers 40 relative to a location of patient 4 and/or applicability of the care provided by care providers 40 to the acute health event experienced by patient 4.

In examples in which HMS 22 performs an analysis to confirm, verify, or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k- Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMD 10. In such examples, rules configuration service 234 may be configured to develop and maintain rules 196 and rules 84. Rules configuration service 234 may be configured to modify these rules based on event feedback data 254 that indicates whether the detections and confirmations of acute health events by IMD 10, computing device 12, and/or HMS 22 were accurate. Event feedback data 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices.

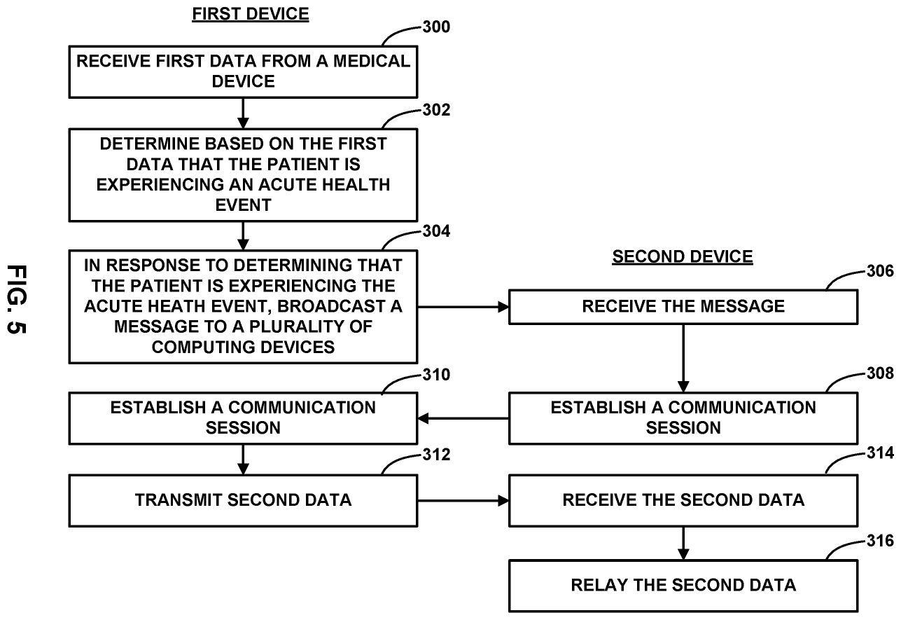

FIG. 5 is a flowchart illustrating examples of the techniques of the present disclosure. The example of FIG. 5 will be described with respect to a first device and a second device. The first device may, for example, be one of computing devices 12 described above, and the second device may be one of computing devices 42 described above.

In the example of FIG. 5, the first device, receives first data from a medical device (300), and determines based on the first data that a user of the first device is experiencing an acute health event (302). In response to determining that the user of the first device is experiencing the acute health event, the first device broadcasts a message to a plurality of devices, which includes the second device (304). The second device receives the message (306), and in response to receiving the message, establishes a communication session with the first device (308). The first device likewise establishes the communication session (310), such that the first device and the second device form an ad hoc wireless network. As part of the communication session, the first device transmits second data to the second device (312). The second device receives the second data (314) and relays the second data to third party service provider (316). The second data may, for example, include encrypted medical information, as well as other information such as a location of the first device, and indication of presence of an arrhythmia, and indication of a type of arrhythmia, an onset time of the arrhythmia, a heart rate detected, a blood perfusion level detected, an indication of patient activity level, a patient posture or position at the time of the acute health event, a blood pressure of the patient, an ambient air temp, any gyroscopic position information of the first device, light levels, sounds levels, or any other such information.

Example 1. A device comprising: communication circuitry configured to communicate between a medical device of a patient and a plurality of computing devices; processing circuitry communicatively coupled to the communication circuitry and configured to: receive, via the communication circuitry, data from the medical device; determine based on the data that the patient is experiencing an acute health event; and in response to determining that the patient is experiencing the acute heath event, broadcast a message to the plurality of computing devices to establish a wireless network with one or more of the plurality of computing devices.

Example 2. The device of Example 1, wherein the processing circuitry is further configured to: determine that a network connection is unavailable; and broadcast the message to the plurality of computing devices in response to determining that the network connection is unavailable.

Example 3. The device of Example 2, wherein to determine that the network connection is unavailable, the processing circuitry is configured to determine that one or both of an Internet connection or a cellular network connection is unavailable.

Example 4. The device of any of Examples 1-3, wherein to broadcast the message to the plurality of computing devices, the processing circuitry is configured to cause the communication circuitry to broadcast the message using a Bluetooth protocol.

Example 5. The device of any of Examples 1-4, wherein the message includes a universally unique identifier (UUID) for the device.

Example 6. The device of any of Examples 1-5, wherein the processing circuitry is further configured to: establish a communication session with a second device and communicate over the wireless network via the second device.

Example 7. The device of any of Examples 1-6, wherein the processing device is configured to send, via the communication circuitry, encrypted medical information to the second device.

Example 8. The device of any of Examples 1-7, wherein the medical device comprises an implantable medical device.

Example 9. The device of any of Examples 1-8, wherein the wireless network is established with the one or more of the plurality of computing devices to facilitate an acute care response by one or more responders who are physically proximate to the patient.

Example 10. The device of any of Examples 1-9, wherein the wireless network is established with the one or more of the plurality of computing devices to automatically share patient data in compliance with patient data privacy constraints to one or more responders who are physically proximate to the patient in order to facilitate an acute care response by the responders.

Example 11. A method for operating processing circuitry of a medical device: receiving data from the medical device; determining based on the data that a patient is experiencing an acute health event; and in response to determining that the patient is experiencing the acute heath event, broadcast a message to the plurality of computing devices to establish a wireless network with one or more of the plurality of computing devices.

Example 12. The method of Example 11, the method further comprising: determining that a network connection is unavailable; and broadcasting the message to the plurality of computing devices in response to determining that the network connection is unavailable.

Example 13. The method of Example 12, wherein determining that the network connection is unavailable comprises determining that one or both of an Internet connection or a cellular network connection is unavailable.

Example 14. The method of any of Examples 11-13, wherein broadcasting the message to the plurality of computing devices comprises broadcasting the message using a Bluetooth protocol.

Example 15. The method of any of Examples 11-14, wherein the message includes a universally unique identifier (UUID) for the device.

Example 16. The method of any of Examples 11-15, further comprising: establishing a communication session with a second device; communicating over the wireless network via the second device.

Example 17. The method of any of Examples 11-16, further comprising: sending encrypted medical information to the second device.

Example 18. The method of any of Examples 11-17, wherein the medical device comprises an implantable medical device.

Example 19. A computer readable medium storing instructions that when executed by one or more processors cause the one or more process to perform the method of any of Examples 9-16.

Example 20. A system comprising: a first device configured to: receive data from a medical device; determine based on the data that the patient is experiencing an acute health event; and in response to determining that the patient is experiencing the acute heath event, broadcast a message to a plurality of computing devices; and a second device, wherein the second device belongs to the plurality of devices and the second device is configured to: receive the message from the first device; establish a communication session with the first device in response to receiving the message.

Example 21. The system of Example 20, wherein to establish the communication session with the first device, the second device is configured to establish an ad hoc, wireless network with one or more of the plurality of computing devices.

Example 22. The system of Example 20 or 21, wherein the first device is further configured to: determine that a network connection is unavailable; and broadcast the message to the plurality of computing devices in response to determining that the network connection is unavailable.

Example 23. The system of Example 22, wherein to determine that the network connection is unavailable, the first device is configured to determine that one or both of an Internet connection or a cellular network connection is unavailable.

Example 24. The system of any of Examples 20-23, wherein to broadcast the message to the plurality of computing devices, the first device is configured to broadcast the message using a Bluetooth protocol.

Example 25. The system of any of Examples 20-24, wherein the message includes a universally unique identifier (UUID) for the device.

Example 26. The system of Example 25, wherein the second device is configured to listen for the UUID and in response to receiving the UUID from the first device, establish the communication session with the first device.

Example 27. The system of any of Examples 20-26, wherein the first device is configured, during the communication session, to transmit encrypted medical information to the second device, and the second device is configured to transmit the encrypted medical information to a third party using one or both of an Internet connection or a cellular network connection.

Example 29. The system of any of Examples 20-27, wherein the medical device comprises an implantable medical device.

Example 30. The system of any of Examples 20-28, wherein the communication session is established with the second device to facilitate an acute care response by a user of the second device.

Example 31. The system of any of Examples 20-29, wherein the communication session is established with the second device to automatically share patient data in compliance with patient data privacy constraints to one or more responders who are physically proximate to the patient in order to facilitate an acute care response by the responders.

Example 32. The system of any of Examples 20-31, wherein the messages is broadcast within a range of less than 150 meters of the first device.

Example 33. The system of any of Examples 20-31, wherein the messages is broadcast within a range of less than 40 meters of the first device.

Example 34. The system of any of Examples 20-31, wherein the messages is broadcast within a range of less than 275 meters of the first device.

Example 35. The system of any of Examples 20-31, wherein the messages is broadcast within a range of less than 125 meters of the first device.

Example 36. The system of any of Examples 20-31, wherein the messages is broadcast within a range of at least 15 meters of the first device.

Example 37. The system of any of Examples 2-31, wherein the messages is broadcast within a range of 1-100 meters of the first device.

Example 38. A first device configured to communicate with a second device, the first device comprising: communication circuitry configured to receive a message from the second device; processing circuitry communicatively coupled to the communication circuitry and configured to: receive, via the communication circuitry, data from the medical device; determine based on the data that the patient is experiencing an acute health event; and in response to receiving the message, establish a wireless network for transmission of medical information with the second device.

Example 39. The first device of Example 38, wherein the processing circuitry is further configured to output an alert in response to receiving the message.

Example 40. The device of Example 38 or 39, wherein to receive the message, the communication circuitry is configured to receive a Bluetooth pairing request.

Example 41. The device of any of Examples 38-40, wherein the message includes a universally unique identifier (UUID) for the second device, and the processing circuitry is configured to establish the wireless network for transmission of the medical information with the second device in response to recognizing the UUID as a UUID reserved for emergency medical communication.

Example 42. The device of any of Examples 38-41, wherein the processing circuitry is configured to receive, via the communication circuitry, encrypted medical information from the second device.

Example 43. The device of any of Examples 38-42, wherein the wireless network is established with the second device to facilitate an acute care response by one or more responders who are physically proximate to the patient.

Example 44. The device of any of Examples 38-42, wherein the wireless network is established with the second device to automatically share patient data from the second device in compliance with patient data privacy constraints to one or more responders who are physically proximate to the patient in order to facilitate an acute care response by the responders.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

1. A device comprising:
communication circuitry configured to communicate between a medical device of a patient and a plurality of computing devices;
processing circuitry communicatively coupled to the communication circuitry and configured to:
receive, via the communication circuitry, data from the medical device;
determine based on the data that the patient is experiencing an acute health event; and
in response to determining that the patient is experiencing the acute heath health event, broadcast a message to the plurality of computing devices to establish a wireless network with one or more of the plurality of computing devices.

2. The device of claim 1, wherein the processing circuitry is further configured to:
determine that a network connection is unavailable; and
broadcast the message to the plurality of computing devices in response to determining that the network connection is unavailable.

3. The device of claim 2, wherein to determine that the network connection is unavailable, the processing circuitry is configured to determine that one or both of an Internet connection or a cellular network connection is unavailable.

4. The device of claim 1, wherein to broadcast the message to the plurality of computing devices, the processing circuitry is configured to cause the communication circuitry to broadcast the message using a Bluetooth protocol.

5. The device of claim 1, wherein the message includes a universally unique identifier (UUID) for the device.

6. The device of claim 1, wherein the processing circuitry is further configured to:
establish a communication session with a second device and communicate over the wireless network via the second device.

7. The device of claim 6, wherein the processing circuitry is configured to send, via the communication circuitry, encrypted medical information to the second device.

8. The device of claim 1, wherein the medical device comprises an implantable medical device.

9. The device of claim 1, wherein the wireless network is established with the one or more of the plurality of computing devices to facilitate an acute care response by one or more responders who are physically proximate to the patient.

10. The device of claim 1, wherein the wireless network is established with the one or more of the plurality of computing devices to automatically share patient data in compliance with patient data privacy constraints to one or more responders who are physically proximate to the patient in order to facilitate an acute care response by the responders.

11. A method for operating processing circuitry of a medical device:
receiving data from the medical device;
determining based on the data that a patient is experiencing an acute health event; and
in response to determining that the patient is experiencing the acute heath health event, broadcast a message to the plurality of computing devices to establish a wireless network with one or more of the plurality of computing devices.

12. The method of claim 11, the method further comprising:
determining that a network connection is unavailable; and
broadcasting the message to the plurality of computing devices in response to determining that the network connection is unavailable.

13. A system comprising:
a first device configured to:
receive data from a medical device;
determine based on the data that a patient is experiencing an acute health event; and
in response to determining that the patient is experiencing the acute health event, broadcast a message to a plurality of computing devices; and
a second device, wherein the second device belongs to the plurality of computing devices and the second device is configured to:
receive the message from the first device; and
establish a communication session with the first device in response to receiving the message.

14-15. (canceled)

16. The system of claim 13, wherein to establish the communication session with the first device, the second device is configured to establish an ad hoc, wireless network with one or more of the plurality of computing devices.

17. The system of claim 13, wherein the first device is further configured to:
determine that one or both of an Internet connection or a cellular network connection is unavailable; and
broadcast the message to the plurality of computing devices in response to determining that the one or both of the Internet connection or the cellular network connection is unavailable.

18. The system of claim 13, wherein to broadcast the message to the plurality of computing devices, the first device is configured to broadcast the message using a Bluetooth protocol.

19. The system of claim 13, wherein the message includes a universally unique identifier (UUID) for the first device, and wherein the second device is configured to listen for the UUID and in response to receiving the UUID from the first device, establish the communication session with the first device.

20. The system of any of claim 13, wherein the first device is configured, during the communication session, to transmit encrypted medical information to the second device, and the second device is configured to transmit the encrypted medical information to a third party using one or both of an Internet connection or a cellular network connection.

21. The method of claim 11, further comprising:
sending encrypted medical information to a second device, wherein the second device belongs to the plurality of computing devices.

22. The method of claim 11, wherein the medical device comprises an implantable medical device.
